(19) United States
(12) Patent Application Publication
(10) Pub. No.: US 2013/0195973 A1
(43) Pub. Date: Aug. 1, 2013

(54) EXTENDED RELEASE PHARMACEUTICAL DOSAGE FORMS OF CARBIDOPA AND LEVODOPA AND PROCESS OF PREPARATION THEREOF

(71) Applicant: RANBAXY LABORATORIES LIMITED, New Delhi (IN)

(72) Inventors: Sweta GUPTA, Ghaziabad (IN); Shavej AHMAD, Lucknow (IN); Romi Barat SINGH, Varanasi (IN)

(73) Assignee: RANBAXY LABORATORIES LIMITED, New Delhi (IN)

(21) Appl. No.: 13/752,909

(22) Filed: Jan. 29, 2013

(30) Foreign Application Priority Data
Jan. 30, 2012 (IN) 254/DEL/2012

(51) Int. Cl. A61K 31/198

(52) U.S. Cl. CPC A61K 31/198
USPC 424/452; 514/565

(57) ABSTRACT

The present invention relates to an extended-release pharmaceutical dosage form of carbidopa and levodopa comprising (i) an immediate-release unit of carbidopa and levodopa; (ii) an extended-release unit of carbidopa and levodopa; and (iii) an immediate or extended-release unit of a carboxylate salt. The present invention further provides a process of preparation thereof.

### FIELD OF THE INVENTION

The present invention relates to an extended-release pharmaceutical dosage form of carbidopa and levodopa comprising (i) an immediate-release unit of carbidopa and levodopa; (ii) an extended-release unit of carbidopa and levodopa; and (iii) an immediate or extended-release unit of a carboxylate salt. The present invention further provides the process of preparation thereof.

### BACKGROUND OF THE INVENTION

Parkinson's disease is associated with the depletion of dopamine from cells in the corpus striatum. Since dopamine does not cross the blood-brain barrier and cannot therefore be used to treat Parkinson's disease, its immediate precursor, levodopa, is used instead because it penetrates the brain where it is decarboxylated to dopamine. But levodopa is also decarboxylated to dopamine in peripheral tissues and consequently only a small portion of administered levodopa is transported unchanged to the brain. This reaction can be blocked by carbidopa which inhibits decarboxylation of peripheral levodopa but cannot itself cross the blood-brain barrier and has no effect on the metabolism of levodopa in the brain.

The combination of carbidopa and levodopa is considered to be the most effective treatment and has been used for several years for treating symptoms of Parkinson's disease. Currently this combination is commercially available as immediate-release Sinemet® and as controlled-release Sinemet® CR tablets.

Nevertheless, certain limitations become apparent within two to five years of initiating combination therapy. As the disease progresses, the benefit from each dose becomes shorter (“the wearing-off effect”) and some patients fluctuate unpredictably between mobility and immobility (“the on-off effect”). “On” periods are usually associated with high plasma levodopa concentrations and often include abnormal involuntary movements. “Off” periods have been correlated with low plasma levodopa concentrations and bradykinetic episodes.

Currently available controlled-release formulations of carbidopa and levodopa reduce these effects to some extent by continuously releasing the drug over a prolonged period of time.

However, there remains a significant problem with currently available controlled-release formulations of carbidopa and levodopa, i.e., the considerable delay in onset of action. The patients face this problem particularly in the morning when symptoms appear, because most of the last evening's dose wears off during the night. Now, patients suffering from symptoms in the morning are unwilling to take a controlled-release formulation with a delayed onset of action. In such cases, a controlled-release dosage form that could also provide rapid onset of action, at least equivalent to that of conventional carbidopa-levodopa, would have an obvious clinical advantage over current therapy.

U.S. Patent Application No. 2010/0298268 discloses a novel carbidopa and levodopa dosage form that provides constant or relatively steady levodopa plasma concentrations over a prolonged period of time to optimize relief of symptoms with immediate onset of action.

However, there remains a need in the art to develop other alternative dosage forms of carbidopa and levodopa which provide steady levodopa plasma concentration with immediate onset of action, which in turn reduces the “the wearing-off effect” and “the on-off effect”.

The inventors have now developed an extended-release pharmaceutical dosage form of carbidopa and levodopa which provides constant or steady levodopa plasma concentration over a prolonged period of time with decreased motor fluctuations, reduced “off” time, and increased “on” time in patients.

### SUMMARY OF THE INVENTION

Hence, in one general aspect, there is provided an extended-release pharmaceutical dosage form of carbidopa and levodopa comprising:
(i) an immediate-release unit of carbidopa and levodopa;
(ii) an extended-release unit of carbidopa and levodopa; and
(iii) an immediate-release unit of a carboxylate salt.

In another general aspect, there is provided an extended-release pharmaceutical dosage form of carbidopa and levodopa comprising:
(i) an immediate-release unit of carbidopa and levodopa;
(ii) an extended-release unit of carbidopa and levodopa; and
(iii) an immediate-release unit of a carboxylate salt

wherein both (i) and (ii) units comprise carbidopa and levodopa in a weight ratio of about 1:1 to about 1:10.

According to one of the embodiments, the immediate-release unit of carbidopa and levodopa comprises carbidopa, levodopa and one or more pharmaceutically acceptable excipients.

According to another embodiment, the extended-release unit of carbidopa and levodopa comprises carbidopa, levodopa, one or more release-controlling agents, and one or more pharmaceutically acceptable excipients.

According to another embodiment, the immediate-release unit of a carboxylate salt comprises a carboxylate salt and one or more pharmaceutically acceptable excipients.

In another general aspect, there is provided an extended-release pharmaceutical dosage form of carbidopa and levodopa comprising:
(i) an immediate-release unit of carbidopa and levodopa;
(ii) an extended-release unit of carbidopa and levodopa; and
(iii) an extended-release unit of a carboxylate salt.

In another general aspect, there is provided an extended-release pharmaceutical dosage form of carbidopa and levodopa comprising:
(i) an immediate-release unit of carbidopa and levodopa;
(ii) an extended-release unit of carbidopa and levodopa; and
(iii) an extended-release unit of a carboxylate salt

wherein both (i) and (ii) units comprise carbidopa and levodopa in a weight ratio of about 1:1 to about 1:10.

According to another embodiment, the immediate-release unit of carbidopa and levodopa comprises carbidopa, levodopa, and one or more pharmaceutically acceptable excipients.

According to another embodiment, the extended-release unit of carbidopa and levodopa comprises carbidopa, levodopa, one or more release-controlling agents, and one or more pharmaceutically acceptable excipients.

According to another embodiment, the extended-release unit of a carboxylate salt comprises a carboxylate salt, one or more release-controlling agents, and one or more pharmaceutically acceptable excipients.

According to another embodiment, the extended-release units of the dosage form are prepared as a reservoir type, matrix type, or a combination thereof. Reservoir type dosage forms utilize a coating of release-controlling agents over the core of the drug; matrix type dosage forms are those in which the drug is distributed uniformly within rate-controlling agents. A combination of reservoir type and matrix type includes release-controlling coatings over extended-release matrices.

The immediate-release and extended-release units of the dosage form include spheroids, beads, microspheres, seeds, granules, pellets, mini tablets, ion-exchange resin beads, and other multi-particulate systems.

The units are prepared using any pharmaceutically acceptable techniques known in the art such as wet granulation, dry granulation, direct compression, extrusion-spheronization, or hot melt extrusion.

According to another embodiment, the extended-release units of the dosage form comprise release-controlling agents to form the reservoir.

According to another embodiment, all three units are mixed in a particular ratio so as to provide the desired in-vitro and in-vivo release profiles.

According to another embodiment, all three units are mixed and either filled in a capsule or compressed into tablet dosage form.

In another general aspect, the extended-release pharmaceutical dosage form of carbidopa and levodopa is prepared by a process comprising the steps of:
(i) preparing the immediate-release unit comprising carbidopa, levodopa, and one or more pharmaceutically acceptable excipients;
(ii) preparing the extended-release unit comprising carbidopa, levodopa, one or more release-controlling agents, and one or more pharmaceutically acceptable excipients;
(iii) preparing the immediate-release unit comprising salt of carboxylic acid and one or more pharmaceutically acceptable excipients; and
(iv) mixing all three units in a particular ratio and filling into a capsule.

In another aspect, there is provided a process for preparing the extended-release pharmaceutical dosage form of carbidopa and levodopa wherein the process comprises the steps of:
(i) preparing the immediate-release unit comprising carbidopa, levodopa, and one or more pharmaceutically acceptable excipients;
(ii) preparing the extended-release unit comprising carbidopa, levodopa, one or more release-controlling agents, and one or more pharmaceutically acceptable excipients;
(iii) preparing the extended-release unit comprising salt of carboxylic acid, one or more release-controlling agents, and one or more pharmaceutically acceptable excipients; and
(iv) mixing all three units in a particular ratio and filling into a capsule.

In another general aspect, there is provided a method of treating symptoms of Parkinson's disease in a mammal by administering an extended-release pharmaceutical dosage form of carbidopa and levodopa comprising:
(i) an immediate-release unit of carbidopa and levodopa;
(ii) an extended-release unit of carbidopa and levodopa; and
(iii) an immediate-release unit of a carboxylate salt.

In another general aspect, there is provided a method of treating symptoms of Parkinson's disease in a mammal by administering an extended-release pharmaceutical dosage form of carbidopa and levodopa comprising:
(i) an immediate-release unit of carbidopa and levodopa;
(ii) an extended-release unit of carbidopa and levodopa; and
(iii) an extended-release unit of a carboxylate salt.

In another embodiment, the extended-release pharmaceutical dosage form of the present invention may further include one or more drugs to treat Parkinson's disease such as entacapone, pramipexole, rasagiline, selegiline, ropinirole, piribedil, bromocriptine, pergolide, lisuride, cabergoline, apomorphine, rotigotine, tolcapone, and amantadine.

### DETAILED DESCRIPTION OF THE INVENTION

The term “unit”, as used herein, includes spheroids, beads, microspheres, seeds, granules, pellets, mini tablets, ion-exchange resin beads, and other multi-particulate systems.

The term “immediate-release unit”, as used herein, refers to a unit which releases carbidopa and levodopa substantially immediately upon administration with complete dissolution within an hour.

The term “extended-release unit”, as used herein, refers to a unit which releases carbidopa, levodopa, or a carboxylate salt over a prolonged period of time, i.e., over a period of more than an hour.

The term “carboxylate salt”, as used herein, refers to a carboxylate with a base. The carboxylate has at least one carboxyl group and may be a mono, di, or polycarboxylic acid depending upon the number of carboxyl groups present. Carboxylic acids react with bases to form salts, in which the hydrogen atom of the hydroxyl group of carboxylic acid is replaced with a metal cation of a base. The salts include but are not limited to, alkaline earth metal salts such as sodium, potassium, lithium, calcium, strontium, barium, and antimony; ammonium salts; and amine or alkanolamine salts of the carboxylic acid. Examples of carboxylic acids include, but are not limited to, tartaric acid, adipic acid, succinic acid, citric acid, benzoic acid, maleic acid, acetic acid, ascorbic acid, edetic acid, fumaric acid, lactic acid, malic acid, oleic acid, sorbic acid, stearic acid, palmitic acid, glutaric acid, glutamic acid, mandelic acid, or mixtures thereof. The carboxylate salt of the present invention preferably includes sodium citrate or sodium tartarate. When more than one carboxylic acid group is present, there may be attached two different cations such as a double salt, e.g. Rochelle salt or sodium potassium tartarate.

The term “release-controlling agent” includes, but is not limited to cellulose derivatives such as ethyl cellulose, butyl cellulose, hydroxypropyl methyl cellulose, hydroxypropyl cellulose, ethyl hydroxyethyl cellulose, carboxymethyl cellulose, cellulose acetate butyrate, cellulose acetate phthalate, hydroxypropyl methylcellulose phthalate, hydroxypropyl methylcellulose acetate succinate, cellulose acetate, and cellulose propionate; vinyl polymers such as polyvinyl chloride, polyvinyl acetate, polyvinyl butyrate, polyvinyl alcohol, polyvinyl pyrrolidones, (e.g., Plasdone® K-90), homopolymer of N-vinyl-2-pyrrolidone, polyvinyl acetate phthalates, N-vinyl-2-pyrrolidone, and polyvinyl ester; polysaccharides such as alginate, xanthan, carrageenan, scleroglucan, pullulan, dextran, haluronic acid, chitin, chitosan, and starch; other natural polymers like proteins (e.g. albumin, gelatin); natural rubber; synthetic polymers such as acrylates, such as polymethacrylate, poly(hydroxy ethyl methacrylate), poly(methyl methacrylate), and poly(hydroxy ethyl methacrylate-co methyl methacrylate); neutral methacrylic polymers such as Eudragit® FS 30 D, Eudragit® S 100, and Eudragit® L 100; methacrylate copolymers with trimethyl-aminoethyl-methacrylate such as Eudragit® RL, Eudragit® RS, Eudragit® NE, Carbopol 934, ethyl acrylate-methyl methacrylate-ethyl ammonium trimethyl chloride methacrylate copolymer, and acrylic acid type polymer having a quaternary ammonium-alkyl group; polyamides such as polyacrylamide and poly(methylene bisacrylamide); polyanhydrides such as poly(bis carboxyphenoxy)methane; PEO-PPO block-co-polymers such as poloxamers; polyethylene glycols and co-polymers thereof; polyethylene oxides and co-polymers thereof; polypropylene and co-polymers thereof; polystyrene; polyesters such as poly(lactic acid), poly(glycolic acid), poly(caprolactone), and co-polymers thereof; poly(ortho esters) and co-polymers thereof; resins such as Dowex™ and Amberlite™; polycarbonate; cellophane; silicones such as poly(dimethylsiloxane); shellacs; waxes such as carnauba wax, beeswax, glycowax, and castor wax; stearates such as glycerol palmitostearate, glyceryl monostearate, glyceryl tristearate, and stearyl alcohol; lipids such as glycerides and phospholipids; paraffin; and cetyl alcohol, or mixtures thereof.

The release-controlling agent may either be present in the matrix or in the coating. The release-controlling agent may be present in a concentration from about 5% to about 30% w/w of the total dosage form.

The extended-release unit comprising carbidopa and levodopa is coated with a coating composition with percentage weight gain of about 5% to about 40% w/w.

The extended-release unit comprising salt of carboxylic acid is coated with a coating composition with a percentage weight gain of about 5% to about 40% w/w.

The release-controlling agent used in the present invention particularly comprises one or more pH-dependent polymers such as methacrylic acid copolymers, e.g., Eudragit® L 100 and Eudragit® S 100, which dissolve at pH 6.0 and 7.0, respectively. Eudragit® L 100 and S 100 are copolymers of methacrylic acid and methyl methacrylate, respectively. The ratio of free carboxyl groups to ester groups is approximately 1:1 in Eudragit® L 100 and 1:2 in Eudragit® S 100.

Eudragit® L 100 is a pH dependent anionic co-polymer powder derived from methacrylic acid and methyl methacrylate with a ratio of free carboxy groups to ester groups of approximately 1:1 and solubilizing above pH 6.0 for targeted drug delivery in the jejunum. Preferably, it has a mean molecular weight of greater than about 100,000 daltons, e.g. about 135,000 daltons. It is commercially available from Evonik Industries.

Eudragit® S 100 is a pH dependent anionic copolymer derived from methacrylic acid and methyl methacrylate with a ratio of free carboxy groups to ester groups of approximately 1:2 and solubilizing above pH 7.0 for targeted drug delivery in the upper bowel. Preferably, it has a mean molecular weight of greater than about 100,000 daltons, e.g. about 135,000 daltons. It is currently commercially available from Evonik Industries.

The term “dosage form” of the present invention includes tablets or capsules. All three units remain distinct and separate from each other. The three units can be filled in a capsule dosage or can be compressed together to form a trilayer tablet; the units may be present in either order.

The immediate-release and extended-release units of carbidopa and levodopa of the present invention comprise carbidopa and levodopa in a weight ratio of about 1:1 to about 1:10, preferably in a weight ratio of 1:4.

The extended-release pharmaceutical dosage form of the present invention comprises from about 25 mg to about 2000 mg levodopa, preferably from about 50 mg to about 600 mg of levodopa.

The extended-release pharmaceutical dosage form of the present invention comprises from about 10 mg to about 300 mg carbidopa, preferably from about 10 mg to about 80 mg of carbidopa.

The term “pharmaceutically acceptable excipient”, as used herein, includes solubility enhancers, fillers, binders, lubricant/glidants, coloring agents, plasticizers, and opacifiers.

Specific examples of solubility enhancers include polyethylene glycols, surfactants, propylene glycol, glycerol, mono-alcohols, higher alcohols, DMSO, dimethylformamide, N-dimethylacetamide, 2-pyrolidone, N-(2-hydroxyethyl) pyrrolidone, N-methylpyrrolidone, 1-dodecylazacycloheptan-2-one, other N-substituted-alkyl azacycloalkyl-2-ones, or mixtures thereof.

Specific examples of fillers or diluents include lactose, pregelatinized starch, calcium carbonate, calcium phosphate dibasic, calcium phosphate tribasic, calcium sulphate, kaolin, starch, or mixtures thereof.

Specific examples of lubricants/glidants include colloidal silicon dioxide, stearic acid, magnesium stearate, calcium stearate, zinc stearate, sodium stearyl fumarate, talc, hydrogenated castor oil, sucrose esters of fatty acid, microcrystalline wax, yellow beeswax, white beeswax, or mixtures thereof.

Specific examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, or mixtures thereof.

Coloring agents include any FDA approved color for oral use.

Specific examples of plasticizers include triethylcitrate, dibutylsebacate, acetylated triacetin, tributylcitrate, glyceroltributyrate, monoglyceride, rapeseed oil, olive oil, sesame oil, acetyltributylcitrate, acetyltriethylcitrate, glycerin sorbitol, diethyloxalate, diethyl phthalate, diethylmalate, diethylfumarate, dibutylsuccinate, diethylmalonate, dioctylphthalate, or mixtures thereof.

Specific examples of opacifiers include titanium dioxide, manganese dioxide, iron oxide, silicon dioxide, or mixtures thereof.

In another embodiment of the present invention, the extended-release pharmaceutical dosage form may be prepared by conventional techniques known in the art such as wet granulation, dry granulation, direct compression, extrusion-spheronization, or hot melt extrusion. The wet granulation process involves the use of water or any other suitable granulating fluid. The dry granulation may involve the use of roller compacter or chilsonator.

The coating layers may comprise one or more film-forming polymers and coating additives.

Specific examples of granulating fluid/solvents for coating include acetone, ethanol, isopropyl alcohol, methylene chloride, or combinations thereof.

Coating additives may be selected from the group consisting of plasticizers, coloring agents, and lubricants/glidants.

Examples of film-forming polymers include ethylcellulose, hydroxypropyl methylcellulose, hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit®; and the like. Alternatively, commercially available coating compositions comprising film-forming polymers marketed under various trade names, such as Opadry®, may also be used.

Examples of plasticizers include triethylcitrate, acetylated triacetin, tributylcitrate, glyceroltributyrate, monoglyceride, rapeseed oil, olive oil, sesame oil, acetyltributylcitrate, acetyltriethylcitrate, glycerin sorbitol, diethyloxalate, diethyl phthalate, diethylmalate, diethylfumarate, dibutylsuccinate, diethylmalonate, dioctylphthalate, dibutylsebacate, and the like.

Coating may be performed by applying the coating composition as a solution/suspension/blend using any conventional coating technique known in the prior art, such as spray coating in a conventional coating pan or fluidized bed processor; dip coating; or compression coating.

The invention may be further illustrated by the following examples, which are for illustrative purposes only and should not be construed as limiting the scope of the invention in any way.

### Example 1

### Mini Tablets of Immediate-Release Unit of Carbidopa Levodopa

| Ingredients | Percent w/w (%) |
|---|---|
| Carbidopa | 14.29 |
| Levodopa | 57.14 |
| Microcrystalline cellulose | 14.29 |
| Hydroxypropyl cellulose | 7.14 |
| Magnesium stearate | 7.14 |
| Purified water | q.s. |
| Total | 100.00 |

### Example 1A

### Mini Tablets of Extended-Release Unit of Carbidopa and Levodopa

| | |
|---|---|
| Ingredients | Percent w/w (%) |

Core

| | |
|---|---|
| Carbidopa | 11.90 |
| Levodopa | 47.61 |
| Microcrystalline cellulose | 11.90 |
| Hydroxypropyl cellulose | 5.95 |
| Magnesium stearate | 5.95 |
| Purified water | q.s. |

Extended-Release Coating

| | |
|---|---|
| Eudragit ® L 100 | 14.16 |
| Triethyl citrate | 1.66 |
| Talc | 0.83 |
| Ethanol | q.s. |
| Total | 100.00 |

### Example 1B

### Mini Tablets of Extended-Release Unit of Carbidopa and Levodopa

| | |
|---|---|
| Ingredients | Percent w/w (%) |

Core

| | |
|---|---|
| Carbidopa | 11.90 |
| Levodopa | 47.61 |
| Microcrystalline cellulose | 11.90 |
| Hydroxypropyl cellulose | 5.95 |
| Magnesium stearate | 5.95 |
| Purified water | q.s. |

Extended-Release Coating

| | |
|---|---|
| Eudragit ® S 100 | 14.16 |
| Triethyl citrate | 1.66 |
| Talc | 0.83 |
| Ethanol | q.s. |
| Total | 100.00 |

### Example 1C

### Mini Tablets of Extended-Release Unit of Carbidopa and Levodopa

| | |
|---|---|
| Ingredients | Percent w/w (%) |

Core

| | |
|---|---|
| Carbidopa | 11.90 |
| Levodopa | 47.61 |
| Microcrystalline cellulose | 11.90 |
| Hydroxypropyl cellulose | 5.95 |
| Magnesium stearate | 5.95 |
| Purified water | q.s. |

Extended-Release Coating

| | |
|---|---|
| Hydroxypropyl methyl cellulose (Hypromellose 55) | 14.16 |
| Diacetylated monoglyceride | 1.66 |
| Talc | 0.83 |
| Ethanol | q.s. |
| Total | 100.00 |

### Example 1D

### Mini Tablets of Extended-Release Unit of Carbidopa and Levodopa

| | |
|---|---|
| Ingredients | Percent w/w (%) |

Core

| | |
|---|---|
| Carbidopa | 11.90 |
| Levodopa | 47.61 |
| Microcrystalline cellulose | 11.90 |
| Hydroxypropyl cellulose | 5.95 |
| Magnesium stearate | 5.95 |
| Purified water | q.s. |

Extended-Release Coating

| | |
|---|---|
| Eudragit ® L 100 | 7.08 |
| Eudragit ® S 100 | 7.08 |
| Triethyl citrate | 1.66 |
| Talc | 0.83 |
| Ethanol | q.s. |
| Total | 100.00 |

### Example 1E

### Mini Tablets of Extended-Release Unit of Carbidopa and Levodopa

| | |
|---|---|
| Ingredients | Percent w/w (%) |

Core

| | |
|---|---|
| Carbidopa | 11.90 |
| Levodopa | 47.61 |
| Microcrystalline cellulose | 11.90 |
| Hydroxypropyl cellulose | 5.95 |
| Magnesium stearate | 5.95 |
| Purified water | q.s. |

Extended-Release Coating

| | |
|---|---|
| Eudragit ® L 100 | 9.44 |
| Eudragit ® S 100 | 4.72 |
| Triethyl citrate | 1.66 |
| Talc | 0.83 |
| Ethanol | q.s. |
| Total | 100.00 |

### Example 1F

### Mini Tablets of Extended-Release Unit of Carbidopa and Levodopa

| | |
|---|---|
| Ingredients | Percent w/w (%) |

Core

| | |
|---|---|
| Carbidopa | 12.98 |
| Levodopa | 51.94 |
| Microcrystalline cellulose | 12.98 |
| Hydroxypropyl cellulose | 6.49 |
| Magnesium stearate | 6.49 |
| Purified water | q.s. |

Extended-Release Coating

| | |
|---|---|
| Eudragit ® L 100 | 5.15 |
| Eudragit ® S 100 | 2.58 |
| Triethyl citrate | 0.91 |
| Talc | 0.45 |
| Ethanol | q.s. |
| Total | 100.00 |

### Example 1G

### Mini Tablets of Immediate-Release Unit of Carboxylate Salt

| Ingredients | Percent w/w (%) |
|---|---|
| Sodium citrate | 71.43 |
| Microcrystalline cellulose | 14.29 |
| Hydroxypropyl cellulose-L | 7.14 |
| Magnesium stearate | 7.14 |
| Purified water | q.s. |
| Total | 100.00 |

### Example 1H

### Mini Tablets of Immediate-Release Unit of Carboxylate Salt

| Ingredients | Percent w/w (%) |
|---|---|
| Sodium tartarate | 71.43 |
| Microcrystalline cellulose | 14.29 |
| Hydroxypropyl cellulose-L | 7.14 |
| Magnesium stearate | 7.14 |
| Purified water | q.s. |
| Total weight | 100 |

### Example 1I

### Mini Tablets of Extended-Release Unit of Carboxylate Salt

| | |
|---|---|
| Ingredients | Percent w/w (%) |

Core

| | |
|---|---|
| Sodium citrate | 59.52 |
| Microcrystalline cellulose | 11.90 |
| Hydroxypropyl cellulose-L | 5.95 |
| Magnesium stearate | 5.95 |
| Purified water | q.s. |

Extended-Release Coating

| | |
|---|---|
| Eudragit ® L100 | 14.16 |
| Triethyl citrate | 1.66 |
| Talc | 0.83 |
| Ethanol | q.s. |
| Total | 100.00 |

### Example 1J

### Mini Tablets of Extended-Release Unit of Carboxylate Salt

| | |
|---|---|
| Ingredients | Percent w/w (%) |

Core

| | |
|---|---|
| Sodium citrate | 59.52 |
| Microcrystalline cellulose | 11.90 |
| Hydroxypropyl cellulose-L | 5.95 |
| Magnesium stearate | 5.95 |
| Purified water | q.s. |

Extended-Release Coating

| | |
|---|---|
| Eudragit ® S 100 | 14.16 |
| Triethyl citrate | 1.66 |
| Talc | 0.83 |
| Ethanol | q.s. |
| Total | 100.00 |

### Example 1K

### Mini Tablets of Extended-Release Unit of Carboxylate Salt

| | |
|---|---|
| Ingredients | Percent w/w (%) |

Core

| | |
|---|---|
| Sodium citrate | 59.52 |
| Microcrystalline cellulose | 11.90 |
| Hydroxypropyl cellulose-L | 5.95 |
| Magnesium stearate | 5.95 |
| Purified water | q.s. |

Extended-Release Coating

| | |
|---|---|
| Hydroxypropyl methyl cellulose (Hypromellose-55) | 14.16 |
| Diacetyl monoglyceride | 1.66 |
| Talc | 0.83 |
| Ethanol | q.s. |
| Total | 100.00 |

### Example 1L

### Mini Tablets of Extended-Release Unit of Carboxylate Salt

| | |
|---|---|
| Ingredients | Percent w/w (%) |

Core

| | |
|---|---|
| Sodium tartarate | 59.52 |
| Microcrystalline cellulose | 11.90 |
| Hydroxypropyl cellulose-L | 5.95 |
| Magnesium stearate | 5.95 |
| Purified water | q.s. |

Extended-Release Coating

| | |
|---|---|
| Eudragit ® L 100 | 7.08 |
| Eudragit ® S 100 | 7.08 |
| Triethyl citrate | 1.66 |
| Talc | 0.83 |
| Ethanol | q.s. |
| Total | 100.00 |

### Example 1M

### Mini Tablets of Extended-Release Unit of Carboxylate Salt

| | |
|---|---|
| Ingredients | Percent w/w (%) |

Core

| | |
|---|---|
| Sodium tartrate | 59.52 |
| Microcrystalline cellulose | 11.90 |
| Hydroxypropyl cellulose-L | 5.95 |
| Magnesium stearate | 5.95 |
| Purified water | q.s. |

Extended-Release Coating

| | |
|---|---|
| Eudragit ® L 100 | 9.44 |
| Eudragit ® S 100 | 4.72 |
| Triethyl citrate | 1.66 |
| Talc | 0.83 |
| Ethanol | q.s. |
| Total | 100.00 |

### Example 1N

### Mini Tablets of Extended-Release Unit of Carboxylate Salt

| | |
|---|---|
| Ingredients | Percent w/w (%) |

Core

| | |
|---|---|
| Sodium tartrate | 64.92 |
| Microcrystalline cellulose | 12.98 |
| Hydroxypropyl cellulose-L | 6.49 |
| Magnesium stearate | 6.49 |
| Purified water | q.s. |

Extended-Release Coating

| | |
|---|---|
| Eudragit ® L 100 | 5.15 |
| Eudragit ® S 100 | 2.58 |
| Triethyl citrate | 0.91 |
| Talc | 0.45 |
| Ethanol | q.s. |
| Total | 100.00 |

### Procedure

### Example 1A

### Mini Tablet of Immediate-Release Unit of Carbidopa Levodopa

1. Levodopa, carbidopa, and microcrystalline cellulose are sifted through a suitable sieve;
2. The premix of step 1 is mixed in a Rapid Mixer Granulator (RMG);
3. The binder solution of hydroxypropyl cellulose in purified water is prepared;
4. The blend of step 2 is granulated with the binder solution of step 3;
5. The granules of step 4 are dried and sifted through a suitable sieve;
6. Magnesium stearate is sifted through a suitable sieve and is mixed with the granules of step 5;
7. The lubricated blend of step 6 is compressed using a suitable tooling.

### Examples 1B-1F

### Mini Tablets of Extended-Release Unit of Carbidopa Levodopa

1. Levodopa, carbidopa, and microcrystalline cellulose are sifted through a suitable sieve;
2. The premix of step 1 is mixed in a RMG;
3. The binder solution of hydroxypropyl cellulose in purified water is prepared;
4. The blend of step 2 is granulated with the binder solution of step 3;
5. The granules of step 4 are dried and sifted through a suitable sieve;
6. Magnesium stearate is sifted through a suitable sieve and is mixed with the granules of step 5;
7. The lubricated blend of step 6 is compressed using a suitable tooling;
8. Eudragit® L 100 of Example 1A, Eudragit® S 100 of Example 1B, hypromellose of Example 1C, or combinations of Eudragit® L 100 and Eudragit® S 100 of Examples 1D-1F are dissolved in ethanol under continuous stirring;
9. Triethyl citrate of Examples 1A, 1B, and 1D-1F and diacetylated monoglyceride of Example 1C are added to the solution of step 8;
10. Talc is dispersed into the solution of step 9;
11. Core tablets of step 7 are coated with the coating solution of step 10 to get desired tablet weight.

### Examples 1G-1H

### Mini Tablet of Immediate-Release Unit of Carboxylate Salt

1. Sodium citrate of Example 1G or sodium tartarate of Example 1H are mixed with microcrystalline cellulose and are sifted through a suitable sieve;
2. The premix of step 1 is mixed in a RMG;
3. The binder solution of hydroxypropyl cellulose in purified water is prepared;
4. The blend of step 2 is granulated with the binder solution of step 3;
5. The granules of step 4 are dried and sifted through a suitable sieve;
6. Magnesium stearate is sifted through a suitable sieve and is mixed with the granules of step 5;
7. The lubricated blend of step 6 is compressed using a suitable tooling.

### Examples 1I-1N

### Mini Tablets of Extended-Release Unit of Carboxylate Salt

1. Sodium citrate of Examples 1I, 1J, and 1K or sodium tartrate of Examples 1L, 1M, and 1N is mixed with microcrystalline cellulose and sifted through a suitable sieve;
2. The premix of step 1 is mixed in a RMG;
3. The binder solution of hydroxypropyl cellulose in purified water is prepared;
4. The blend of step 2 is granulated with the binder solution of step 3;
5. The granules of step 4 are dried and sifted through a suitable sieve;
6. Magnesium stearate is sifted through a suitable sieve and is mixed with the granules of step 5;
7. The lubricated blend of step 6 is compressed using a suitable tooling;
8. Eudragit® L 100 of Example 1H, Eudragit® S 100 of Example 1I, hypromellose of Example 1J, or combinations of Eudragit® L 100 and Eudragit® S 100 of Examples 1K-1M are dissolved in ethanol under continuous stirring;
9. Triethyl citrate of Examples 1H, 1I, and 1K-1M or diacetylated monoglyceride of Example 1J are added to the solution of step 8;
10. Talc is dispersed into the solution of step 9;
11. Core tablets of step 7 are coated with coating solution of step 10 to get desired tablet weight.

### Capsule Filling:

The mini tablets prepared by following above four procedures are filled into capsules depending upon the desired-release profile.

### Example 2

### Mini Tablet of Immediate-Release Unit of Carbidopa and Levodopa

| Ingredients | Percent w/w (%) |
|---|---|
| Carbidopa | 15.40 |
| Levodopa | 57.30 |
| Microcrystalline cellulose | 13.00 |
| Hydroxypropyl cellulose | 7.10 |
| Magnesium stearate | 7.10 |
| Purified water | q.s. |
| Total | 100.00 |

### Mini Tablets of Extended-Release Unit of Carbidopa and Levodopa

| | |
|---|---|
| Ingredients | Percent w/w (%) |

Core

| | |
|---|---|
| Carbidopa | 13.90 |
| Levodopa | 51.70 |
| Microcrystalline cellulose | 11.70 |
| Hydroxypropyl cellulose | 6.40 |
| Magnesium stearate | 6.40 |
| Purified water | q.s. |

Extended-Release Coating

| | |
|---|---|
| Eudragit ® L 100 | 4.20 |
| Eudragit ® S 100 | 4.20 |
| Triethyl citrate | 1.00 |
| Talc | 0.50 |
| Isopropyl alcohol | q.s. |
| Total | 100.00 |

### Mini Tablets of Extended-Release Unit of Carboxylate Salt

| | |
|---|---|
| Ingredients | Percent w/w (%) |

Core

| | |
|---|---|
| Sodium citrate | 63.80 |
| Microcrystalline cellulose | 12.80 |
| Hydroxypropyl cellulose-L | 6.40 |
| Magnesium stearate | 6.40 |
| Purified water | q.s. |

Extended-Release Coating

| | |
|---|---|
| Eudragit ® L 100 | 4.50 |
| Eudragit ® S 100 | 4.50 |
| Triethyl citrate | 1.10 |
| Talc | 0.50 |
| Isopropyl alcohol | q.s. |
| Total | 100.00 |

### Procedure

### Mini Tablet of Immediate-Release Unit of Carbidopa Levodopa

1. Levodopa, carbidopa, microcrystalline cellulose, and hydroxypropyl cellulose were sifted through a suitable sieve;
2. The premix of step 1 was mixed in a Rapid Mixer Granulator (RMG);
3. The mixture of step 2 was granulated using purified water;
4. The granules of step 3 were dried and sifted through a suitable sieve;
5. Magnesium stearate was sifted through a suitable sieve and was mixed with the granules of step 4;
6. The lubricated blend of step 5 was compressed using a suitable tooling into mini tablets.

### Mini Tablets of Extended-Release Unit of Carbidopa Levodopa

7. Levodopa, carbidopa, microcrystalline cellulose, and hydroxypropyl cellulose were sifted through a suitable sieve;
8. The premix of step 7 was mixed in a Rapid Mixer Granulator (RMG);
9. The mixture of step 8 was granulated using purified water;
10. The granules of step 9 were dried and sifted through a suitable sieve;
11. Magnesium stearate was sifted through a suitable sieve and was mixed with the granules of step 10;
12. The lubricated blend of step 11 was compressed using a suitable tooling into mini tablets;
13. Eudragit® L 100 and Eudragit® S 100 were dissolved in an isopropyl alcohol and water mixture under continuous stirring;
14. Triethyl citrate and talc were dispersed in an isopropyl alcohol and water mixture under continuous stirring;
15. The dispersion of step 14 was added to the solution of step 13 under continuous stirring to form the coating solution;
16. The mini tablets of step 12 were coated with the coating solution of step 15 to form coated mini tablets.

### Mini Tablets of Extended-Release Unit of Carboxylate Salt

17. Sodium citrate, microcrystalline cellulose, and hydroxypropyl cellulose were sifted through a suitable sieve;
18. The premix of step 17 was mixed in a Rapid Mixer Granulator (RMG);
19. The mixture of step 18 was granulated using purified water;
20. The granules of step 19 were dried and sifted through a suitable sieve;
21. Magnesium stearate was sifted through a suitable sieve and was mixed with the granules of step 20;
22. The lubricated blend of step 21 was compressed using a suitable tooling into mini tablets;
23. Eudragit® L 100 and Eudragit® S 100 were dissolved in an isopropyl alcohol and water mixture under continuous stirring;
24. Triethyl citrate and talc were dispersed in an isopropyl alcohol and water mixture under continuous stirring;
25. The dispersion of step 24 was added to the solution of step 23 under continuous stirring to form the coating solution;
26. The mini tablets of step 22 were coated with the coating solution of step 25 to form coated mini tablets.

### Capsule Filling:

27. One mini tablet of step 6, four mini tablets of step 16, and two mini tablets of step 26 were filled into a capsule.

We claim:

1. An extended-release pharmaceutical dosage form of carbidopa and levodopa comprising:
(i) an immediate-release unit of carbidopa and levodopa;
(ii) an extended-release unit of carbidopa and levodopa; and
(iii) an immediate-release unit of a carboxylate salt.

2. An extended-release pharmaceutical dosage form of carbidopa and levodopa comprising:
(i) an immediate-release unit of carbidopa and levodopa;
(ii) an extended-release unit of carbidopa and levodopa; and
(iii) an extended-release unit of a carboxylate salt.

3. The extended-release pharmaceutical dosage form of claims 1 and 2, wherein the carboxylate salt is selected from the group consisting of sodium, potassium, lithium, calcium, strontium, barium, antimony, ammonium, amine or alkanolamine salts of tartaric acid, adipic acid, succinic acid, citric acid, benzoic acid, maleic acid, acetic acid, ascorbic acid, edetic acid, fumaric acid, lactic acid, malic acid, oleic acid, sorbic acid, stearic acid, palmitic acid, glutaric acid, glutamic acid, mandelic acid, or mixtures thereof.

4. The extended-release pharmaceutical dosage form of claim 3, wherein the carboxylate salt is sodium citrate.

5. The extended-release pharmaceutical dosage form of claims 1 and 2, wherein the immediate and extended-release units of carbidopa and levodopa comprise carbidopa and levodopa in a weight ratio of about 1:1 to about 1:10.

6. The extended-release pharmaceutical dosage form of the preceding claims, wherein the extended-release unit comprises release-controlling agents selected from the group consisting of cellulose derivatives, vinyl polymers, polysaccharides, other natural polymers, synthetic polymers, resins, shellacs, waxes, stearates, lipids, or a mixture thereof.

7. The extended-release pharmaceutical dosage form of the preceding claims, wherein the dosage form further comprises solubility enhancers, fillers, binders, lubricant, coloring agents, plasticizers, and opacifiers.

8. A process for preparing the extended-release pharmaceutical dosage form of carbidopa and levodopa wherein the process comprises the steps of:
(i) preparing the immediate-release unit comprising carbidopa, levodopa, and one or more pharmaceutically acceptable excipients;
(ii) preparing the extended-release unit comprising carbidopa, levodopa, one or more release-controlling agents, and one or more pharmaceutically acceptable excipients;
(iii) preparing the immediate-release unit comprising a carboxylate salt and one or more pharmaceutically acceptable excipients.

9. A process for preparing the extended-release pharmaceutical dosage form of carbidopa and levodopa wherein the process comprises the steps of:
(i) preparing the immediate-release unit comprising carbidopa, levodopa, and one or more pharmaceutically acceptable excipients;
(ii) preparing the extended-release unit comprising carbidopa, levodopa, one or more release-controlling agents, and one or more pharmaceutically acceptable excipients;
(iii) preparing the extended-release unit comprising a carboxylate salt, one or more release-controlling agents, and one or more pharmaceutically acceptable excipients.

10. The process of claim 8 or 9 further comprising the step of:
(iv) mixing the units of steps (i) to (iii) in a particular ratio and filling into a capsule.

11. The process of claim 8 or 9 further comprising the step of:
(iv) mixing the units of steps (i) to (iii) and compressing them into a tablet.

12. The method of treating symptoms of Parkinson's disease in a mammal by administering an extended-release pharmaceutical dosage form of carbidopa and levodopa comprising an immediate-release unit of carbidopa and levodopa, an extended-release unit of carbidopa and levodopa, and an immediate-release or extended-release unit of a carboxylate salt.
